(19) United States
(12) Patent Application Publication
(10) Pub. No.: US 2004/0166520 A1
(43) Pub. Date: Aug. 26, 2004

(54) Identifying items with nucleic acid taggants

(76) Inventor: D. Michael Connolly, Rochester, NY (US)

Correspondence Address: Nixon Peabody LLP, Clinton Square, P.O. Box 31051, Rochester, NY, 14603-1051, US

(21) Appl. No.: 10/743,991

(22) Filed: Dec. 23, 2003

Related U.S. Application Data
(60) Provisional application No. 60/438,265, filed on Jan. 3, 2003.

(51) Int. Cl. C12Q 1/68

(52) U.S. Cl. 435/6

(57) ABSTRACT

The present invention provides a method of identifying a tagged item which involves recovering from the item a nucleic acid containing taggant sample potentially containing one or more target nucleic acids. A detection unit is provided having multiple electrically separated electrical conductor pairs. Each conductor pair has an attached capture probe such that a gap exists between the capture probes of a pair of electrically separated conductors, and the capture probes for each pair of conductors are complementary to one of the target nucleic acids. The sample is contacted with the detection unit under conditions effective to permit any target nucleic acid in the sample to bind to the capture probes, thereby connecting the capture probes. Target nucleic acid in the sample is detected by determining whether electricity is conducted between the electrically separated conductors. Also provided is a kit for identification of a nucleic acid taggant on an item.

This application claims the benefit of U.S. Provisional Patent Application Serial No. 60/438,265 filed Jan. 3, 2003.

### FIELD OF THE INVENTION

The present invention relates to a method and a kit for identifying a tagged item by detection of a nucleic acid taggant associated with the item.

### BACKGROUND OF THE INVENTION

Forgery of branded materials has a large impact for companies that lose revenue due to competition from counterfeit products; for customers who may have safety concerns associated with poor quality counterfeit goods; and for governments that may lose tax revenues. Anti-counterfeiting technologies are currently used in every conceivable product including licensed clothing, computers, software, electrical goods, and consumable products. Additionally, the tracking of explosives and materials used to produce explosive devices is a major concern for law enforcement agencies worldwide.

Current anti-counterfeiting technologies include security printing with special watermarks, inks and dyes, holograms, tamper-proof labels, and magnetic and radio frequency identification tags (“RFID tags”). While all these methods are effective to some extent, none is completely counterfeit-proof. In contrast, the incorporation of cloaked DNA (or other nucleic acid) taggants into a product or its packaging provides a virtually counterfeit-proof method of determining the authenticity and source of the material.

Analysis of nucleic acids, such as deoxyribonucleic acids (DNA) or ribonucleic acids (RNA), for clinical and forensic uses has become a routine procedure. For instance, molecular biology techniques allow detection of congenital or infectious diseases based on nucleic acid sequences. These same techniques can also characterize DNA for use in settling factual issues in legal proceedings, such as paternity suits and criminal prosecution. DNA testing has been made possible due to amplification methods. One can take small amounts (theoretically a single molecule) of DNA which, in and of itself, would be undetectable, and increase or amplify the quantity present to a degree where an amount sufficient for detection is present. This amplification has been made possible by the widely used technique known as polymerase chain reaction (“PCR”).

U.S. Pat. No. 5,139,812 to Lebacq describes a method of secretly marking moveable property with a small amount of a known DNA molecule. The DNA can be that of the owner of the property. Provided the mark is made in secret, and is not visible, proof of ownership can be established by amplifying the DNA, reading the DNA sequence of base pairs, and showing that it corresponds with that of the owner. However, it would be relatively simple for a counterfeiter to apply a mark of another DNA to the property.

U.S. Pat. No. 6,312,911 to Bancroft et al., describes a method of DNA-based steganography, wherein a DNA sequence corresponding to a coded message is inserted into genomic DNA. Because of the complexity and size of genomic DNA, there is no good way to determine which portion of the genome is the message, unless one possesses the key, i.e., the knowledge of where to look for the message.

Although it is relatively simple to tag a product or its packaging with a mark or label consisting of a nucleic acid such as DNA, the marking is only useful as a tracking/anti-counterfeiting tool if it can be read easily and quickly by authorized personnel in a secure manner. The major flaw in current DNA-taggant systems is that the equipment needed to read the code is not portable, requires skilled operators, and takes hours or even days to provide a response.

The present invention is directed to overcoming these and other deficiencies in the art.

### SUMMARY OF THE INVENTION

The present invention relates to a method of identifying a tagged item. This involves recovering a nucleic acid containing taggant sample from an item, where the taggant sample potentially contains one or more target nucleic acids. A detection unit is provided having one or more sets of electrically separated electrical conductor pairs. Each conductor has an attached capture probe such that a gap exists between the capture probes of a pair of electrically separated conductors. The capture probes for each pair of separated electrical conductors are complementary to one of the target nucleic acids. The sample is contacted with the detection unit under conditions effective to permit any target nucleic acid present in the taggant sample to bind to the capture probes. As a result, the capture oligonucleotides are connected. Any target nucleic acid present in the taggant sample is detected by determining whether electricity is conducted between the electrically separated conductors. This identifies the tagged item.

The present invention also relates to nucleic acid taggant identification kit. The kit includes a nucleic acid-containing taggant, having one or more target nucleic acids, and a detection cartridge. The detection cartridge has one or more sets of electrically separated electrical conductor pairs. Each conductor has an attached capture probe such that a gap exists between the capture probes of a pair of electrically separated conductors. The capture probes for each pair of separated electrical conductors are complementary to one of the target nucleic acids.

The advantages of the method provided by the present invention over those methods currently available are: 1) individual users can determine their own tagging code, providing flexibility and security; 2) detection analysis is rapid; and 3) detection can be performed on-site by a non-technical operator. Samples do not need to be sent to a laboratory for analysis by trained personnel. A consignment of merchandise can be checked at any point during shipping and a definitive determination of its authenticity made 15-30 minutes after sampling.

The use of DNA as a property marker has been previously proposed (for example, U.S. Pat. No. 5,139,812 to Lebacq). However, the routine use of oligonucleotides, such as DNA for tagging of merchandise, is dependent on the ability to examine the taggant quickly and easily without having to send samples to a laboratory for analysis. Current state-of-the-art DNA detection technologies rely on the use of fluorescent dyes or radioactive tags to identify hybrid formation. However, thousands of binding events are needed before these signals are detectable, and, therefore, a DNA amplification process is generally required. Amplification processes require a skilled technician and are cumbersome, error prone, and slow. In addition, such systems are not readily portable and cannot simultaneously detect multiple taggants.

### BRIEF DESCRIPTION OF THE DRAWINGS

FIGS. 1A-B show a perspective view of a system for detection of a target nucleic acid molecule from a sample which includes a desk-top detection unit and a detection cartridge which is inserted into the desk-top unit. FIG. 1C shows a schematic view of this system.

FIG. 2 shows a single test unit positioned in the first chamber of a detection cartridge of the present invention used for the detection of a target nucleic acid molecule.

FIGS. 3A-B show a perspective view of a system for detection of a target nucleic acid molecule which includes a portable detection unit and a detection cartridge which is inserted into the portable unit. FIG. 3C shows a schematic view of this system.

FIGS. 4A-G show exemplary applications of the nucleic acid taggant identification method of the present invention. FIG. 4A shows a nucleic acid tag printed onto the surface of a product's packaging. FIG. 4B shows marking by hand with a pen containing nucleic acid tagged ink. FIG. 4C shows a nucleic acid taggant incorporated into packaging material. FIG. 4D shows a clothing label impregnated with a nucleic acid taggant. FIG. 4E shows a bar code label to which a nucleic acid taggant has been applied. FIG. 4F shows a tamper-evident label containing an RNA taggant which degrades when the package is opened. FIG. 4G shows the addition of a nucleic acid taggant to the contents of a drug capsule.

### DETAILED DESCRIPTION OF THE INVENTION

The present invention relates to a method of identifying a tagged item. This involves recovering a nucleic acid containing taggant sample from an item, where the taggant sample potentially contains one or more target nucleic acids. A detection unit is provided having one or more sets of electrically separated electrical conductor pairs. Each conductor has an attached capture probe such that a gap exists between the capture probes of a pair of electrically separated conductors. The capture probes for each pair of separated electrical conductors are complementary to one of the target nucleic acids. The sample is contacted with the detection unit under conditions effective to permit any target nucleic acid present in the taggant sample to bind to the capture probes. As a result, the capture oligonucleotides are connected. Any target nucleic acid present in the taggant sample is detected by determining whether electricity is conducted between the electrically separated conductors. This identifies the tagged item.

The technology described herein relies both on the vast number of possible code combinations that can be achieved with a limited number of nucleic acid tags and on cloaking with irrelevant oligonucleotide fragments to preserve the security of the code. Selecting one or more members of a set of “N” objects can be done in (2^{N}−1) different ways. Thus, a set of 32 different target nucleic acid molecules can yield up to 4,294,967,295 alternative combinations. If the molecules making up the code are mixed with a random selection of similar nucleic acids it will be impossible for a counterfeiter to determine which nucleic acids make up the code.

In one embodiment of the present invention, the unique set of target nucleic acids are 32 different short segments (each having approximately 10 to 30 nucleotide bases) of a nucleotide, such as deoxyribonucleic acid (“DNA”). Alternatively, the oligonucleotides can be ribonucleic acid (“RNA”), peptide nucleic acid (“PNA”), locked nucleic acid (“LNA”) or any other synthetic nucleic acid.

In operation of the present invention, a subset of the 32 target nucleic acid molecules may be selected and mixed with unknown or random nucleic acid molecules. The mixture is then applied to an item of movable property, by methods discussed below. When authentic identification of the item of movable property is desired, the applied mixture of nucleic acid is sampled and analyzed by known methods, as discussed below. The detector reports the presence of any of the 32 known nucleic acid molecules, but does not recognize any others. Thus, if the originally selected set of nucleic acid molecules is present, authenticity is confirmed.

Selection of the code (i.e., a unique subset of identifiable nucleic acid molecules) by the user, combined with a detector which recognizes the entire set of molecules, is an added security feature. In one aspect of the present invention, the code is set by the company manufacturing the product rather than the company supplying the complete set of oligonucleotides.

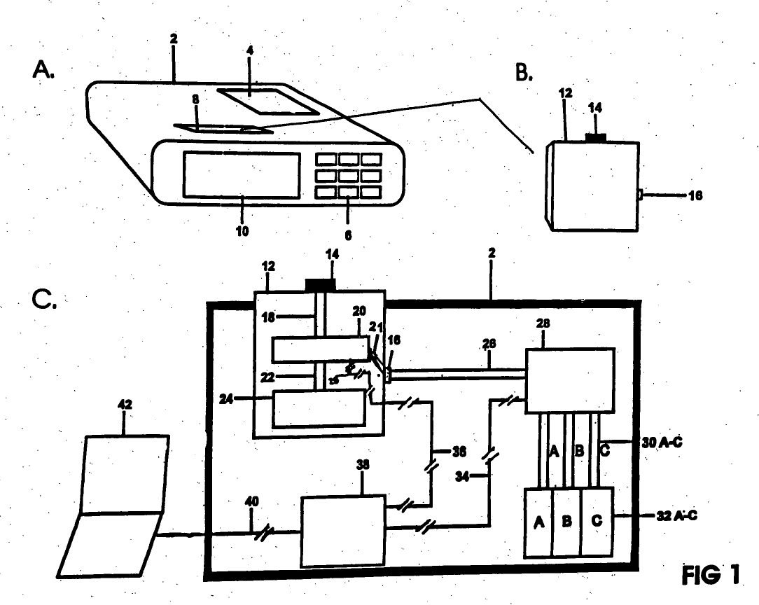

FIGS. 1A-B show a perspective view of a system for detection of a target nucleic acid molecule from a sample. This system includes a desk-top detection unit and a detection cartridge which is inserted into the desk-top unit. In this embodiment, desk-top detection unit 2 is provided with door 4 for filling reagents, control buttons 6, and visual display 10. Slot 8 in desk-top detection unit 2 is configured to receive detection cartridge 12. Detection cartridge 12 further contains first injection port 14 through which a sample solution can be introduced into a first chamber in cartridge 12 and second injection port 16 through which reagents can be introduced into the first chamber.

FIG. 1C shows a schematic view of the system utilizing desk-top detection unit 2 and detection cartridge 12. In this system, desk-top detection unit 2 contains containers 32A-C suitable for holding reagents and positioned to discharge the reagents into first chamber 20 of detection cartridge 12 through second injection port 16 and conduit 21. Containers 32A-C can, for example, carry a neutralizer, a buffer, a conductive ion solution, and an enhancer. The contents of these containers can be replenished through door 4. This is achieved by making containers 32A-C sealed and disposable or by making them refillable.

Pump 28 removes reagents from containers 32A-C, through tubes 30A-C, respectively, and discharges them through tube 26 and second injection port 16 into detection cartridge 12. Instead of using single pump 28 to draw reagents from containers 32A-C, a separate pump can be provided for each of containers 32A-C so that their contents can be removed individually.

Alternatively, the necessary reagents may be held in containers inside the detection cartridge. The pumps in the detection unit can force a material, such as air, water, or oil, into the detection cartridge to force the reagents from the respective containers and into the first chamber. The reagents are then changed with each detection cartridge, which eliminates the buildup of salt precipitates in the detection unit.

Desk-top detection unit 12 is also provided with controller 38, which is in electrical communication with the electrical conductors of the detection cartridge 12 by means of electrical connector 36, to detect the presence of the target molecule in the sample. Controller 38 also operates pump 28 by way of electrical connector 34. Alternatively, separate controllers can be used for operating the pumps and the detection of target molecules. Digital coupling 40 permits controller 38 to communicate data to computer 42 which is external of desk-top detection unit 12.

Detection cartridge 12 contains first chamber 20 which, as noted supra, receives reagents from within desk-top detection unit 2 by way of second injection port 16 and conduit 21. A sample to be analyzed is discharged to first chamber 20 through first injection port 14 and conduit 18. As described more fully infra, the presence of a target molecule is detected in first chamber 20. Detection cartridge 12 is further provided with second chamber 24 for collecting material discharged from first chamber 20 by way of connector 22. The detection cartridge also contains electrical connector 25 extending through the housing and coupled to the electrically separated conductors in first chamber 20 so that the presence of a target molecule in a sample can be detected.

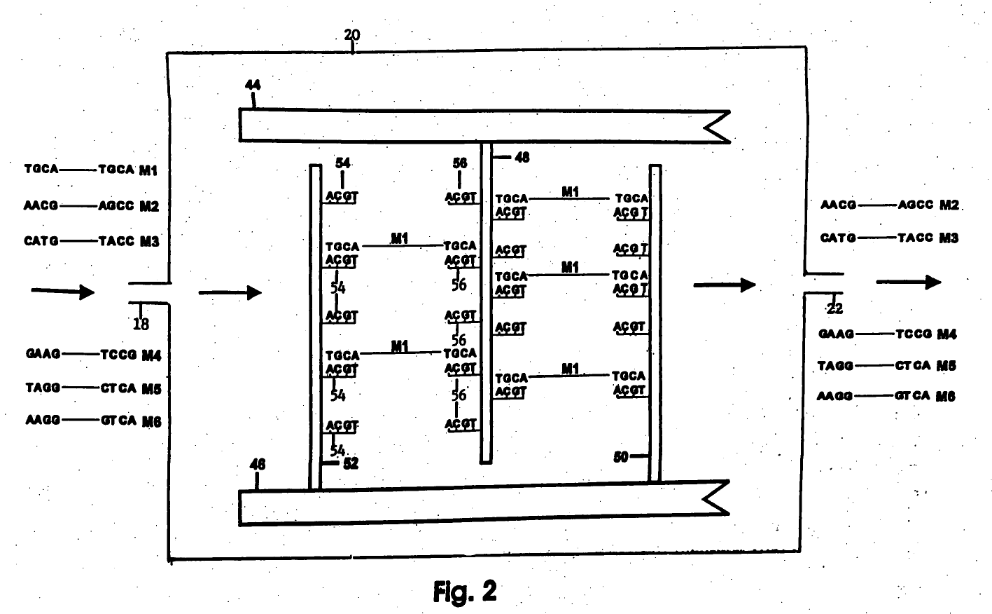

FIG. 2 depicts a single test structure on a detection chip suitable to be positioned in first chamber 20 of the system shown in FIGS. 1A-C. The test structure contains first and second conductive pads 44 and 46, respectively, which are each connected to a source of current and are formed from either the same or different metals. Connected to conductive pads 44 and 46 are electrically separated conductors in the form of spaced apart conductive fingers. Spaced apart conductive fingers 48, 50, and 52 alternatively originate from either of conductive pads 44 and 46, but are not connected to both pads. Short capture probes 54 and 56 are bound to the spaced apart conductive fingers 52 and 48, respectively, in a stable manner. The capture probes can be oligonucleotides or peptide nucleic acid analogs. Capture probes 54 and 56 are separated by sufficient distance to form a gap between the conductive fingers 48, 52, and 56. Typically, the gap will be in microns or fractions of microns in length. Capture probe sequences are complementary to portions of the sequences of the target nucleic acid molecule of interest. Mixtures of nucleic acid molecules (i.e., M1-M6) in a sample enter first chamber 20 through conduit 18 and are washed over the detection chip. Unretained material exits first chamber 20 through connector 22. The sample can either be washed over the chip continuously or incubated for various periods of time on the detection chip.

If a target molecule has a sequence complementary to one of the probes, it can bind to that probe. Once bound to that probe, the molecule is tethered at that site. The sequence complementary to the second probe can then bind to the second probe. To facilitate such a reaction, the two complementary sequences should be chosen such that the length of molecule M1 can span the distance between capture probes 54 and 56. The detection chip is washed to remove any unhybridized nucleic acid molecules (i.e., M2-M6) not captured by capture probes 54 and 56. The nucleic acid molecule complementary to capture probes 54 and 56 (i.e., M1) will be retained on the detection chip and serves to form a nucleic acid connection between conductive fingers 52 and 48, completing an electrical circuit. Here, the electrical conductivity of nucleic acid molecules is relied upon to transmit the electrical signal. Fink et al. reported in “Electrical Conduction through DNA Molecules,” Nature 398(6726):407-10 (1999), which is hereby incorporated by reference in its entirety, that DNA conducts electricity like a semiconductor. This flow of current can be sufficient to construct a simple switch, which will indicate whether or not a target nucleic acid molecule is present within a sample. Optionally, after hybridization of the target nucleic acid molecules to sets of capture probes, the nucleic acid molecules can be coated with a conductor, such as a metal, as described in U.S. Pat. No. 6,664,103 to D. M. Connolly, which is hereby incorporated by reference in its entirety. The coated nucleic acid molecule can then conduct electricity across the gap between the pair of probes, thus producing a detectable signal indicative of the presence of a target nucleic acid molecule. Exemplary metal conductive materials include, without limitation, gold, silver, and mixtures thereof.

The detection chip, on which conductive pads 44 and 46 and conductive fingers 48, 50, and 52 are fixed, is constructed on a support. Examples of useful support materials include, e.g., glass, quartz, and silicon, as well as polymeric substrates, e.g. plastics. In the case of conductive or semi-conductive supports, it will generally be desirable to include an insulating layer on the support. However, any solid support which has a non-conductive surface may be used to construct the device. The support surface need not be flat. In fact, the support may be on the walls of a chamber in a chip.

The detection of a target molecule using a desk-top detection system, as shown in FIGS. 1A-C, can be carried out as follows. After preparation of the sample, the sample is introduced into detection cartridge 12 through first injection port 14 and conduit 18 and into first chamber 20. Once the sample is introduced, detection cartridge 12 is inserted into slot 8 of desk-top detection unit 2 so that second injection port 16 is connected to conduit 21 and electrical connector 36 is coupled to electrical connector 25. The sample is processed in first chamber 20 containing the capture probes and electrical conductors for a period of time sufficient for detection of a target nucleic acid molecule in the sample. Processing of the sample within first chamber 20 can involve neutralizing the sample, contacting the neutralized sample with a buffer, then treating the sample with conductive ions, and treating the sample with an enhancer. Molecules that are not captured are expelled from first chamber 20 through second conduit 22 and into second chamber 24. The desk-top detection system can be programmed by a series of operation buttons 6 on the front of the device and the results can be seen on visual display 10.

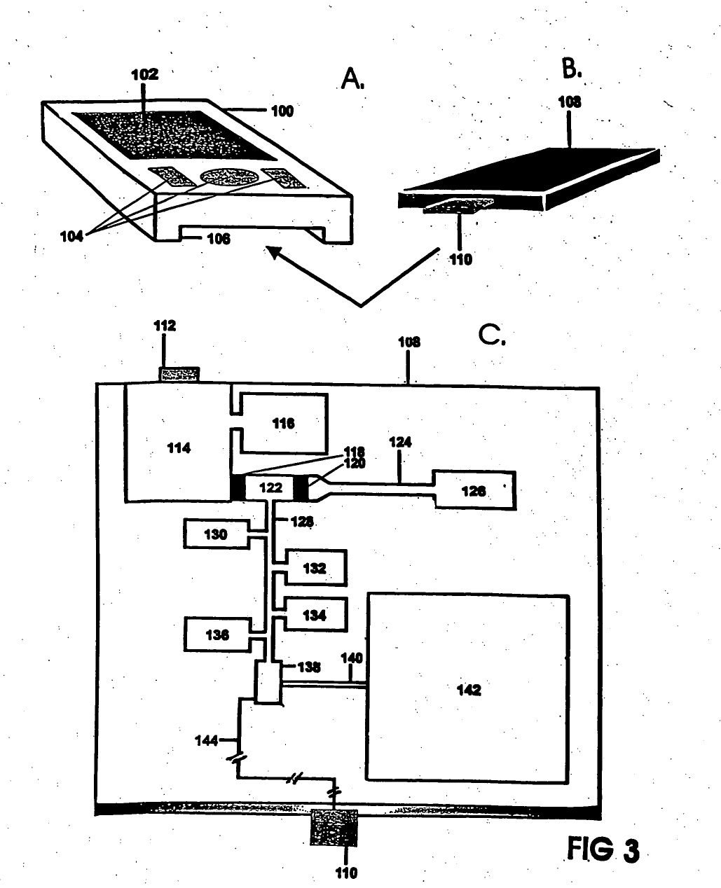

FIGS. 3A-B show a portable detection system. This system is provided with a portable unit 100 which can be in the form of a portable personal digital assistant (e.g., a Palm® unit, 3Com Corporation, Santa Clara, Calif.). Portable unit 100 is provided with visual display 102 and control buttons 104. Slot 106 is provided to receive detection cartridge 108 having electrical connector 110.

FIG. 3C shows a schematic diagram of detection cartridge 108 which is used in the portable detection system of the present invention. Detection cartridge 108 contains first injection port 112 in the housing through which a sample solution can be introduced.

Detection cartridge 108 contains a plurality of containers 130, 132, 134, and 136 suitable for holding reagents and positioned to discharge the reagents into first chamber 138 by way of conduit 128. Containers 130, 132, 134, and 136 can, for example, carry a neutralizer, a buffer, a conductive ion solution, and an enhancer.

Sample pre-treatment chamber 114 is positioned upstream of first chamber 138, and a filter 118 is positioned between pretreatment chamber 114 and first chamber 138. Adjoining pre-treatment chamber 114 is vessel 116 which holds reagents to pre-treat the sample. Detection cartridge 108 also contains pretreatment waste chamber 126 coupled to the pretreatment chamber 114 by way of filter 120 and conduit 124. Second chamber 142 receives material discharged from the first chamber 138 via connector 140. Detection cartridge 108 includes electrical connector 144 which couples the electrically separated conductors in first chamber 138, like those shown in first chamber 20, for the embodiment of FIGS. 1A-C and FIG. 2, to electrical connector 110.

In operation, the detection of a target molecule using a portable detection system, as shown in FIGS. 3A-C, can be carried out as follows. After recovery of the taggant from the item and filtration of the sample containing the recovered taggant, the sample solution is introduced into detection cartridge 108 through first injection port 112. Within sample pretreatment chamber 114, the sample can be pretreated with reagents from first container 116. After denaturation and deproteination, the sample can be concentrated by passing it through filter 118 positioned so that a portion of the pre-treated sample is retained in chamber 122. Excess fluids and unwanted material are passed through filter 120 and waste tube 124 and are collected in pretreatment waste chamber 126. The portion of the sample solution which passes to first chamber 138 is neutralized by the addition of a neutralizer from second container 130. Within first chamber 138, the neutralized target nucleic acid molecule, if present in the sample, is permitted to hybridize with the capture probes on the detection chip in first chamber 138 in substantially the same way as described above with reference to FIGS. 1A-C and FIG. 2. During this period, the contents of first chamber 138 are contacted with a buffer from third container 132. After binding and washing, the sample is treated with a conductive ion solution from fourth container 134, such that conductive ions are deposited on the target molecules that have hybridized to the capture probes on the detection chip. Additionally, after treatment with a conductive ion solution, the sample can be treated with an enhancer solution from fifth container 136 to grow a continuous layer of conductive metal from the deposited conductive ions. Excess buffers and waste buffers will exit first chamber 138 through waste tube 140 and collect in second chamber 142. Electrical connector 144 couples the electrically separated conductors on the detection chip to electrical connector 110 which is connected to portable unit 100. The portable detection system can be programmed by operation of a series of buttons 104 on the front of portable unit 100, and the results are visualized on screen 102.

A plurality of collection methods can be used depending on the type of sample to be analyzed. Liquid samples can be collected by placing a constant volume of the liquid into a lysis buffer. The filter can be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer.

The target nucleic acid whose sequence is to be detected is the identifiable nucleic acid sequence “set” selected by a user to tag an item for identification according to the present invention. A sample of the nucleic acid sequences will be recovered from previously tagged items including: swabs of ink from printed labels, packaging material, the adhesive from a label, or the product itself. Cartridges suitable for use in the device as described herein will be made of plastics that accommodate both organic and inorganic solutes and nucleic acid can be customized to suit a particular user's needs. Extraction of the nucleic acid taggant from the tagged item generally involves dissolving the taggant nucleic acid from the item with aqueous or organic buffers. The dissolvent will not degrade the DNA or interfere with the recognition process. Aqueous buffers include deionized water, various concentrations of buffer compounds, salts, and chaotropic agents. Organic buffers include acetone, dimethylsulfoxide (DMSO), formamide, and the like. In one embodiment of the present invention, removal of the taggant DNA from the item would occur by swabbing the area containing the taggant with the extraction solution. The swab would be placed into a syringe containing hybridization buffer, suitable for subsequence detection. Minimal sample preparation would involve filtering the taggant solution by placing a suitable filter on the end of the syringe. The DNA solution would then be filtered and injected into the DNA detection device using the syringe and an injection port that would mate with the syringe. Authentication would then be carried out by performing the DNA detection process as described. In another embodiment of the present invention, removal of the taggant DNA from the item would occur by removing a small piece of the item containing the DNA taggant and extracting the DNA with hybridization buffer. Minimal sample preparation would involve filtering the taggant solution by placing a suitable filter on the end of the syringe. The DNA solution would then be injected into the DNA detection device using the syringe and an injection port that would mate with the syringe. Authentication would then be carried out by performing the DNA detection process as described. Samples will require minimal pre-processing by filtration to remove particulate debris. The device will identify the presence of known oligonucleotide sequences from samples including: swabs of ink from printed labels, packaging material, the adhesive from a label, or the product itself. Cartridges will be made of plastics that accommodate both organic and inorganic solutes and can be customized to suit a particular user's needs.

Prior to or at the point of contact with the probes, the nucleic acid molecules in the sample are denatured. Denaturation is preferentially carried out by heat treatment. Denaturation can also be carried out by varying the ionic concentration of the carrier solution or by a combination of ionic and heat treatment.

Following extraction, it is often desirable to separate the nucleic acids from other elements of the crude extract, e.g., fibers or particulate matter resulting from the removal of the taggant from an item. Removal of particulate matter is generally accomplished by filtration, flocculation, or the like. Ideally, the sample is concentrated by filtration, which is more rapid and does not require special reagents. A variety of filter types may be readily incorporated into the device. Samples can be forced through filters that will allow only the nucleic acid-containing solution to pass through, trapping debris. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media are also well known in the art and are commercially available from, e.g., Pharmacia (Piscataway, N.J.) and Sigma Chemical (St Louis, Mo.). This isolation and/or gel filtration/desalting may be carried out in an additional chamber, or alternatively, the particular chromatographic media may be incorporated in a channel or fluid passage leading to a subsequent reaction chamber.

The probes are preferably selected to bind with the target such that they have approximately the same melting temperature. This can be done by varying the lengths of the hybridization region. A-T rich regions may have longer target sequences, whereas G-C rich regions would have shorter target sequences.

Hybridization assays on substrate-bound oligonucleotide arrays involve a hybridization step and a detection step. In the hybridization step, the sample potentially containing the target and an isostabilizing agent, denaturing agent, or renaturation accelerant is brought into contact with the probes of the array and incubated at a temperature and for a time appropriate to allow hybridization between the target and any complementary probes.

Including a hybridization optimizing agent in the hybridization mixture significantly improves signal discrimination between perfectly matched targets and single-base mismatches. As used herein, the term “hybridization optimizing agent” refers to a composition that decreases hybridization between mismatched nucleic acid molecules, i.e., nucleic acid molecules whose sequences are not exactly complementary.

An isostabilizing agent is a composition that reduces the base-pair composition dependence of DNA thermal melting transitions. More particularly, the term refers to compounds that, in proper concentration, result in a differential melting temperature of no more than about 1° C. for double stranded DNA oligonucleotides composed of AT or GC, respectively. Isostabilizing agents preferably are used at a concentration between 1 M and 10 M, more preferably between 2 M and 6 M, most preferably between 4 M and 6 M, between 4 M and 10 M, and, optimally, at about 5 M. For example, a 5 M agent in 2×SSPE (Sodium Chloride/Sodium Phosphate/EDTA solution) is suitable. Betaines and lower tetraalkyl ammonium salts are examples of suitable isostabilizing agents.

Betaine (N,N,N,-trimethylglycine; (Rees et al., Biochem. (1993) 32:137-144), which is hereby incorporated by reference in its entirety) can eliminate the base pair composition dependence of DNA thermal stability. Unlike tetramethylammonium chloride (“TMACl”), betaine is zwitterionic at neutral pH and does not alter the polyelectrolyte behavior of nucleic acids while it does alter the composition-dependent stability of nucleic acids. Inclusion of betaine at about 5 M can lower the average hybridization signal, but increases the discrimination between matched and mismatched probes.

A denaturing agent is a composition that lowers the melting temperature of double stranded nucleic acid molecules by interfering with hydrogen bonding between bases in a double-stranded nucleic acid or the hydration of nucleic acid molecules. Denaturing agents can be included in hybridization buffers at concentrations of about 1 M to about 6 M and, preferably, about 3 M to about 5.5 M.

Denaturing agents include formamide, formaldehyde, dimethylsulfoxide (“DMSO”), tetraethyl acetate, urea, guanidine thiocyanate (“GuSCN”), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, and potassium thiocyanate.

A renaturation accelerant is a compound that increases the speed of renaturation of nucleic acids by at least 100-fold. They generally have relatively unstructured polymeric domains that weakly associate with nucleic acid molecules. Accelerants include heterogenous nuclear ribonucleoprotein (“hnRP”) A1 and cationic detergents such as, preferably, cetyltrimethylammonium bromide (“CTAB”) and dodecyl trimethylammonium bromide (“DTAB”), and, also, polylysine, spermine, spermidine, single stranded binding protein (“SSB”), phage T4 gene 32 protein, and a mixture of ammonium acetate and ethanol. Renaturation accelerants can be included in hybridization mixtures at concentrations of about 1 μM to about 10 mM and, preferably, 1 μM to about 1 mM. The CTAB buffers work well at concentrations as low as 0.1 mM.

Addition of small amounts of ionic detergents (such as N-lauryl-sarkosine) to the hybridization buffers can also be useful. LiCl is preferred to NaCl. Hybridization can be at 20°-65° C., usually 37° C. to 45° C. for probes of about 14 nucleotides. Additional examples of hybridization conditions are provided in several sources, including: Sambrook et al., Molecular Cloning: A Laboratory Manual, 2nd Ed., Cold Spring Harbor, N.Y. (1989); and Berger and Kimmel, “Guide to Molecular Cloning Techniques,” Methods in Enzymology Volume 152, Academic Press, Inc., San Diego, Calif. (1987); Young et al., “Efficient Isolation of Genes by Using Antibody Probes,” Proc. Natl Acad. Sci. USA 80(5):1194-8 (1983), which are hereby incorporated by reference in their entirety.

In addition to aqueous buffers, non-aqueous buffers may also be used. In particular, non-aqueous buffers which facilitate hybridization but have low electrical conductivity are preferred. Examples of such buffers include formamide, formaldehyde, dimethylsulfoxide (DMSO) and chaotropic salts.

The sample and hybridization reagents are placed in contact with the array and incubated. Contact can take place in any suitable container, for example, a dish or a cell specially designed to hold the probe array and to allow introduction and removal of fluids. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, hybridization is carried out at a temperature at or between five degrees below the melting temperature and the melting temperature. The target is incubated with the capture probes for a time sufficient to allow the desired level of hybridization between the target and any complementary capture probes. After incubation with the hybridization mixture, the electrically separated conductors are washed with the hybridization buffer, which also can include the hybridization optimizing agent. These agents can be included in the same range of amounts as for the hybridization step, or they can be eliminated altogether.

In one embodiment of the invention, ligation methods may be used to specifically identify single base differences in target nucleic acid sequences. Previously, methods of identifying known target sequences by probe ligation methods have been reported. U.S. Pat. No. 4,883,750 to Whiteley et al.; Wu et al., “The Ligation Amplification Reaction (LAR)—Amplification of Specific DNA Sequences Using Sequential Rounds of Template-Dependent Ligation,” Genomics 4(4):560-569 (1989); Landegren et al., “A Ligase-Mediated Gene Detection Technique,” Science 241(4869):1077-1080 (1988); and Winn-Deen et al., “Sensitive Fluorescence Method for Detecting DNA-Ligation Amplification Products,” Clin. Chem. 37(9):1522-1523 (1991), which are hereby incorporated by reference in their entirety. In one approach, known as oligonucleotide ligation assay (“OLA”), two probes or probe elements which span a target region of interest are hybridized to the target region. Where the probe elements basepair with adjacent target bases, the confronting ends of the probe elements can be joined by ligation, e.g., by treatment with ligase. The ligated probe element is then assayed, evidencing the presence of the target sequence.

In the present invention, one or both probes may be designed to specifically recognize a variation in the sequence at the end of the probe. After the target binds to the probes, the target is treated with nucleases to remove the ends of the molecules which do not bind to the probes. The junction is then treated with ligase. If the complementary sequence is present at the end of the probe, the ligase will ligate the target to the probe. The test chamber can then be heated up to denature non-ligated targets. Detection of the specific target can then be carried out.

Various methods exist for attaching the capture probes to the electrical conductors. For example, U.S. Pat. Nos. 5,861,242, 5,861,242, 5,856,174, 5,856,101, and 5,837,832, which are hereby incorporated by reference in their entirety, disclose a method where light is shone through a mask to activate functional (for oligonucleotides, typically —OH) groups protected with a photo-removable protecting group on a surface of a solid support. After light activation, a nucleoside building block, itself protected with a photo-removable protecting group (at the 5′-OH), is coupled to the activated areas of the support. The process can be repeated, using different masks or mask orientations and building blocks, to place probes on a substrate.

Alternatively, new methods for the combinatorial chemical synthesis of peptide, polycarbamate, and oligonucleotide arrays have recently been reported (see Fodor et al., “Light-Directed, Spatially Addressable Parallel Chemical Synthesis,” Science 251(4995):767-773 (1991); Cho et al., “An Unnatural Biopolymer,” Science 261(5126):1303-1305 (1993); and Southern et al., “Analyzing and Comparing Nucleic Acid Sequences by Hybridization to Arrays of Oligonucleotides: Evaluation Using Experimental Models,” Genomics 13(4):1008-1017 (1992), which are hereby incorporated by reference in their entirety). These arrays (see Fodor et al., “Multiplexed Biochemical Assays with Biological Chips,” Nature 364(6437):555-556 (1993), which is hereby incorporated by reference in its entirety), harbor specific chemical compounds at precise locations in a high-density, information rich format, and are a powerful tool for the study of biological recognition processes.

Preferably, the probes are attached to the leads through spatially directed oligonucleotide synthesis. Spatially directed oligonucleotide synthesis may be carried out by any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide which, itself, optionally has a protected active site if further nucleotide coupling is desired.

In one embodiment, the lead-bound oligonucleotides are synthesized at specific locations by light-directed oligonucleotide synthesis which is disclosed in U.S. Pat. No. 5,143,854, Published PCT Application Serial No. WO 92/10092, and Published PCT Application Serial No. WO 90/15070, which are hereby incorporated by reference in their entirety. In a basic strategy of this process, the surface of a solid support modified with linkers and photolabile protecting groups is illuminated through a photolithographic mask, yielding reactive hydroxyl groups in the illuminated regions. A 3′-O-phosphoramidite-activated deoxynucleoside (protected at the 5′-hydroxyl with a photolabile group) is then presented to the surface and coupling occurs at sites that were exposed to light. Following the optional capping of unreacted active sites and oxidation, the substrate is rinsed and the surface is illuminated through a second mask, to expose additional hydroxyl groups for coupling to the linker. A second 5′-protected, 3′-O-phosphoramidite-activated deoxynucleoside (C-X) is presented to the surface. The selective photodeprotection and coupling cycles are repeated until the desired set of probes are obtained. Photolabile groups are then optionally removed, and the sequence is, thereafter, optionally capped. Side chain protective groups, if present, are also removed. Since photolithography is used, the process can be miniaturized to specifically target leads in high densities on the support.

The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.”

The probes may be targeted to the electrically separated conductors by using a chemical reaction for attaching the probe or nucleotide to the conductor which preferably binds the probe or nucleotide to the conductor rather than the support material. Alternatively, the probe or nucleotide may be targeted to the conductor by building up a charge on the conductor which electrostatically attracts the probe or nucleotide.

Nucleases can be used to remove probes which are attached to the wrong conductor. More particularly, a target nucleic acid molecule may be added to the probes. Targets which bind at both ends to probes, one end to each conductor, will have no free ends and will be resistant to exonuclease digestion. However, probes which are positioned so that the target cannot contact both conductors will be bound at only one end, leaving the molecule subject to digestion. Thus, improperly located probes can be removed while protecting the properly located probes. After the protease is removed or inactivated, the target nucleic acid molecule can be removed and the device is ready for use.

The capture probes can be formed from natural nucleotides, chemically modified nucleotides, or nucleotide analogs, as long as they have activated hydroxyl groups compatible with the linking chemistry. Such RNA or DNA analogs include, but are not limited to, 2′-O-alkyl sugar modifications, methylphosphonate, phosphorothioate, phosphorodithioate, formacetal, 3′-thioformacetal, sulfone, sulfamate, and nitroxide backbone modifications, amides, and analogs, where the base moieties have been modified. In addition, analogs of oligomers may be polymers in which the sugar moiety has been modified or replaced by another suitable moiety, resulting in polymers which include, but are not limited to, polyvinyl backbones (Pitha et al., “Preparation and Properties of Poly (I-vinylcytosine),” Biochim. Biophys. Acta 204(2):381-8 (1970); Pitha et al., “Poly(1-vinyluracil): The Preparation and Interactions with Adenosine Derivatives,” Biochim. Biophys. Acta 204(1):39-48 (1970), which are hereby incorporated by reference in their entirety), morpholino backbones (Summerton, et al., “Morpholino Antisense Oligomers: Design, Preparation, and Properties,” Antisense Nucleic Acid Drug Dev. 7(3): 187-95 (1997), which is hereby incorporated by reference in its entirety) and peptide nucleic acid (PNA) analogs (Stein et al., “A Specificity Comparison of Four Antisense Types: Morpholino, 2′-O-methyl RNA, DNA, and Phosphorothioate DNA,” J. Antisense Nucleic Acid Drug Dev. 7(3):151-7 (1997); Egholm et al., “Peptide Nucleic Acids (PNA)-Oligonucleotide Analogues with an Achiral Peptide Backbone,” (1992); Faruqi et al., “Peptide Nucleic Acid-Targeted Mutagenesis of a Chromosomal Gene in Mouse Cells,” Proc. Natl. Acad. Sci. USA 95(4):1398-403 (1998); Christensen et al., “Solid-Phase Synthesis of Peptide Nucleic Acids,” J. Pept. Sci. 1(3): 175-83 (1995); Nielsen et al., “Peptide Nucleic Acid (PNA). A DNA Mimic with a Peptide Backbone,” Bioconjug. Chem. 5(1):3-7 (1994), which are hereby incorporated by reference in their entirety).

The capture probes can contain the following exemplary modifications: pendant moieties, such as proteins (including, for example, nucleases, toxins, antibodies, signal peptides and poly-L-lysine); intercalators (e.g., acridine and psoralen); chelators (e.g., metals, radioactive metals, boron and oxidative metals); alkylators; and other modified linkages (e.g., alpha anomeric nucleic acids). Such analogs include various combinations of the above-mentioned modifications involving linkage groups and/or structural modifications of the sugar or base for the purpose of improving RNAse H-mediated destruction of the targeted RNA, binding affinity, nuclease resistance, and or target specificity.

Virtually any material or substance used in clothing, or in packaging or labeling goods of all kinds can be marked with a nucleic acid taggant of the present invention, including, without limitation: fabric, paper, cardboard, wood, plastic, nylon, nitrocellulose, rubber, resin, gel, liquid, or adhesive.

In order to make a useful mark, the nucleic acid should be applied in a way that is stable under ambient storage or shipping conditions. For example, if the item being marked is to be left out in the weather, the taggant should be applied in a waterproof matrix. Similar considerations would apply to ensure that the taggant is stable to temperature, pH, humidity, corrosive gases, and electromagnetic radiation.

On the other hand, the nucleic acid should be applied in such a way that it is easy to remove a sample for analysis. For example, this could be done by making the matrix soluble in a solvent contained on a swab. A number of marking methods are described herein, supra.

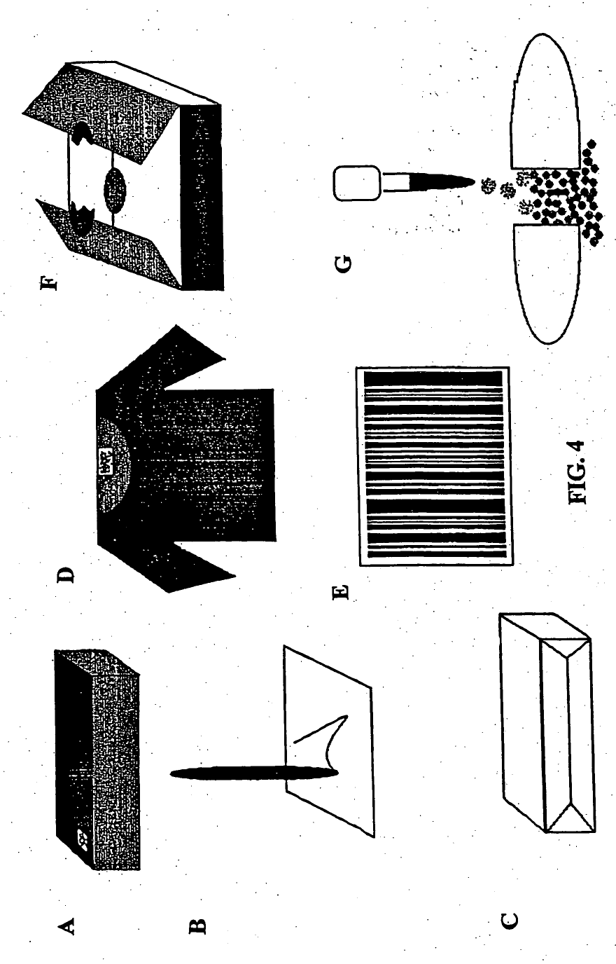

The nucleic acid labeling mixture can be directly printed onto packaging boxes as shown in FIG. 4A, or other articles, using a wide variety of techniques, as shown in FIGS. 4B-G. Nucleic acids can be incorporated into a toner formulation and printed onto the work by electrophotography, or it can be formulated into a water based ink and printed by an inkjet printer. Flexo printing uses a rubber impression cylinder with raised areas where printing is desired. The raised areas are inked, usually with a water based ink, and the ink is transferred to the work piece by light pressure contact.

Printing can also be done with a gravure press, wherein the printing cylinder has indentations corresponding to the printed area. The indentations are filled with ink, and the excess ink is removed with a skiving blade. The printing cylinder is then contacted with the work, and the ink is transferred by capillary action from the cylinder to the work. In the case of a nucleic acid formulated into an oil soluble ink, an offset lithographic press can be used. In this case the printing plate has oleophilic areas where ink is desired, and a hydrophilic background. A water based fountain solution coats the background areas with water, which repels the ink, and the ink coats the oleophilic areas. The ink image is then transferred to an intermediate cylinder, commonly called the blanket cylinder, and from the blanket cylinder to the work.

Other suitable printing methods are: silk screen, where the ink is forced through an image on a fabric carrier by a squeegee and the background portions of the image fabric are blocked by a polymeric photoresist; tampo printing (also known as pad printing), where the image ink is carried on a soft rubber tamp which is pressed onto the workpiece; or pin spot printing, where the image ink is picked up by capillary force into a hollow pin, and then contacted to the work piece where a portion of the ink is deposited.

Nucleic acid ink similar to those used for printing can also be used in a fountain pen, a felt tipped pen, or a ball point pen to mark an item of portable property by hand, as shown in FIG. 4B. Such marks could be visible (if a pigment is added to the nucleic acid mixture) or invisible and could be used to rapidly mark items at point of production. For example, an artist could use such an ink to “sign” a work with a unique signature that could not be forged (the painter, Thomas Kinkade, uses DNA taggant technology to mark his artwork using paint containing his own DNA) or a baseball player could autograph a ball using a nucleic acid-containing ink. Such a rapid marking system could be used to add a further level of security to packaged items (e.g., a covert mark could be superimposed on a pre-printed label or barcode, as shown in FIG. 4E, as an item exits the production facility). The addition of such a mark would require no specialized equipment or expertise and the code could be frequently changed for additional security simply by supplying a new batch of nucleic acid ink.

In the case of porous materials (e.g., wood, cardboard or paper), the oligonucleotide solution could be incorporated into the packaging by: injection with a syringe; infusion; or pressure treatment. Additionally, for both porous and non-porous materials (e.g., plastic film), the oligonucleotides could be incorporated into the packaging material during manufacture, as shown in FIG. 4C. Samples for taggant analysis would be taken either by destroying a small portion of the packaging material, or by using a swab to lift some taggant from the surface. Such a use would make it impossible to remove the tag from the packaging material and would mean that any part of the package could be checked for the presence of the taggant.

In one embodiment of the present invention, a clothing label is imbued with an oligonucleotide solution and allowed to dry, as shown in FIG. 4D. This was used at the Sydney Olympics to prevent import/sale of counterfeit souvenir merchandise. Such a tag is unlikely to survive normal laundering of the clothing, but this is not a problem if the purpose of the taggant is to prevent import/sale of the items rather than prove its identity in the future.

In another embodiment of the present invention, the nucleic acid is dissolved in a solvent such as dimethylformamide along with a water insoluble polymer, such as polyvinylbutryal. When dried, such a mixture will not be soluble in water, and thus can be washed without losing the taggant. For analysis, the nucleic acid extracted by the same solvent can be precipitated by the addition of ethanol, spun down in a centrifuge, and redissolved in buffer for electrophoresis.

Taggants could also be incorporated into paper or plastic labels during their manufacture so that sampling from any part of the label would reveal the taggant or added to the surface after production so that they could be sampled by a swab test (e.g., the barcode label, as shown in FIG. 4E).

Another alternative for marking is to add oligonucleotides to the adhesive used to attach labels to packaging. Traces of such a mark could remain even if the label was removed and would be ideal for incorporation into a tamper-evident label, as shown in FIG. 4F, or as an additional, covert, level of security.

Marker oligonucleotides could be added to the product itself. This would allow any analysis of a sample of the product to show both its identity and source. Such an application would be useful in the case of explosives or of very valuable liquid products such as, drugs, perfume or liquor. In the case of substances which are consumed, a nucleic acid marker has the added advantage of being non-toxic. A nucleic acid taggant could be added to a medicament or its delivery system, including a drug capsule (as shown in FIG. 4G), pill, tablet, lozenge or ointment. A taggant added to products, such as oil or chemicals would also aid in tracing spills as the identity of the taggant would definitively indicate the owner/shipper of the substance spilled and could allow the government to assign responsibility for cleanup costs and/or any appropriate fines. The EPA is currently using microtaggant identification particles consisting of a color code to document the illegal transport and disposal of hazardous and regulated waste (U.S. EPA, 2002), but the oligonucleotide marking system described herein would offer the advantage of rapid on-site identification which is impracticable with the microtaggant system currently in use.

This technology could also be used to make a mark which will show that a package has been opened. In this case, an unstable oligonucleotide (e.g., RNA) is placed within a protective container on the packaging. If the packaging is opened, the integrity of the protective container is breached and the RNA degraded so that it is no longer recognized by the detection system, as shown in FIG. 4F. Such a mark would prevent reuse of legitimate packaging as a cover for counterfeit products and could provide evidence that a product has been tampered with. For example, boxes that originally contained designer jewelry are retrieved for reuse with cheaper counterfeit merchandise. However, if the boxes contained an RNA tag which was destroyed when the original articles were removed, they will no longer be recognized as containing genuine merchandise.

The matrix for the nucleic acid may be a polymeric material. In a preferred embodiment of the present invention, the polymer is polyvinylalcohol, from 88% to 100% hydrolyzed. This polymer is, by virtue of crystalline structure, insoluble in cold water and all organic solvents, but soluble in hot water. Thus, it provides a stable environment for storage of the applied nucleic acid, but can be removed for sampling with hot water on a swab. The polyvinylalcohol can be cross-linked with boric acid to provide even more sample integrity.

Many other polymers can be used as a matrix for a nucleic acid. For example, polyethyleneglycol, polyethyleneimine, polyvinylpyridine, hydroxyethylcellulose, polyvinylbutyral, polyvinylpyrrolidone, polyvinylimidazole, and co-polymers of any of the aforementioned polymers are suitable matrixes for the present invention.

The present invention also relates to a nucleic acid taggant identification kit. The kit of the present invention includes a nucleic acid-containing taggant having one or more target nucleic acids and a detection cartridge. The detection cartridge has one or more sets of electrically separated electrical conductor pairs. Each conductor has an attached capture probe such that a gap exists between the capture probes of a pair of electrically separated conductors. The capture probes for each pair of separated electrical conductors are complementary to one of the target nucleic acids.

In this aspect of the present invention the nucleic-acid containing taggant mixture contains one or more target nucleic acid molecules and is prepared as described above. The nucleic acid taggant mixture is suitable for marking an item for identification. Suitable items for identification using the kit of the present invention include, without limitation, all those described herein above.

In this aspect of the present invention the capture probes for each pair of separated electrical conductors are complementary to one or more of the target nucleic acids provided in the nucleic acid taggant mixture. The method of taggant application, removal, and identification of the taggant sample removed from an item are as described herein above, using the detection cartridge provided in the kit of the present invention. The detection cartridge is suitable for use with the detection unit of the present invention for detecting one or more of the target nucleic acid molecules in a taggant sample removed from an item.

### EXAMPLES

### Example 1

### DNA Detected from Marked Cardboard Box

As a prophetic example, a solution of 12 μl of ladder DNA (Promega, Madison, Wis.) was mixed with 50 μl of 2.5% polyvinylalcohol (98% hydrolyzed). Portions of 15 μl of the solution were spotted onto a glass microscope slide, a cardboard box, and a piece of filter paper and allowed to dry. To simulate rain, the glass microscope slide was run under cold tap water for 10 seconds. After 24 hours, the samples on the microscope slide and the cardboard were recovered by dissolving in 15 μl of warm water. The sample on filter paper was used as is. All three samples were placed in the wells of an agarose electrophoresis gel in TBE buffer. A 70-volt bias was applied to the gel for 2 hours. The gel was stained with Sybr-Gold dye (Molecular Probes, Eugene, Oreg.) and photographed under UV light. Fluorescent bands of the DNA ladder were clearly visible in all three samples and in the control run in the fourth lane. This example shows the utility of marking portable property with samples of DNA and recovering them at a later time to prove the identity of the property.

### Example 2

### DNA Tagging and Detection Using Printing Stamp

As a prophetic example, a tampo printing stamp was prepared by placing a small dab of clear silicone caulking compound (General Electric, Charlotte, N.C.) on a wooded paint stirrer and curing overnight. The top of the caulk dab was sliced off smoothly with a razor blade to create a printing surface. The flat surface of the caulk dab was dipped into a solution of ladder DNA (100 base pair DNA ladder from Promega, Madison, Wis.), and then pressed against a glass microscope slide. The transferred DNA “ink” was allowed to dry. The dry DNA was wiped from the glass slide with a wet cotton swab and the sample removed from the swab by centrifugation at 10,000 rpm for 30 seconds. The DNA ladder was identified by agarose electrophoresis, staining the gel with Sybr-Gold dye (Molecular Probes, Eugene, Oreg.) and observing the DNA bands by fluorescence.

### Example 3

### DNA Tagging of Drug Capsule

As a prophetic example, a capsule of Prozac™ (Eli Lilly, Indianapolis, Ind.) was opened and one microliter of ladder DNA (100 base pair DNA ladder from Promega, Madison, Wis.) was spotted onto the contents of the capsule and allowed to absorb into the drug, as shown in FIG. 4G. The capsule was closed. Although the tagged capsule was visually indistinguishable from un-tagged capsules, the contents could be identified by DNA detection.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of identifying a tagged item comprising:
recovering a nucleic acid containing taggant sample from an item, wherein the taggant sample potentially contains one or more target nucleic acids;
providing a detection unit comprising one or more sets of electrically separated electrical conductor pairs, each conductor having an attached capture probe such that a gap exists between the capture probes of a pair of electrically separated conductors, wherein the capture probes for each pair of separated electrical conductors are complementary to one of the target nucleic acids;
contacting the sample with the detection unit under conditions effective to permit any target nucleic acid present in the taggant sample to bind to the capture probes, thereby connecting the capture probes; and
detecting any target nucleic acid present in the taggant sample by determining whether electricity is conducted between the electrically separated conductors, thereby identifying the tagged item.

2. The method according to claim 1, wherein the taggant sample further comprises random nucleic acid molecules mixed with the target nucleic acids.

3. The method according to claim 1, wherein the target nucleic acids comprise 10 to 30 nucleotides.

4. The method according to claim 3, wherein the target nucleic acids are selected from the group consisting of DNA, RNA, peptide nucleic acids, and locked nucleic acids.

5. The method according to claim 1, wherein the capture probes comprise 10 to 30 nucleotides.

6. The method according to claim 5, wherein the capture probes are selected from the group consisting of DNA, RNA, peptide nucleic acids, and locked nucleic acids.

7. The method according to claim 1, wherein the taggant sample further comprises a matrix.

8. The method according to claim 7, wherein the matrix is selected from the group consisting of polyvinylalcohol, polyethyleneglycol, polyethyleneimine, polyvinylpyridine, hydroxyethylcellulose, polyvinylbutyral, polyvinylpyrrolidone, polyvinylimidazole, and a combination thereof.

9. The method according to claim 1 further comprising:
contacting the capture probes with nucleases after said contacting.

10. The method according to claim 1 further comprising:
contacting the capture probes with ligase after said contacting; and
heating the capture probes to a temperature high enough to denature any non-ligated target nucleic acids from the capture probes.

11. The method according to claim 1 further comprising:
applying a conductive material over the capture probes and any target nucleic acid after said contacting.

12. The method according to claim 11, wherein the conductive material is selected from the group consisting of gold, silver, and mixtures thereof.

13. The method according to claim 1, wherein the taggant sample is stable under ambient conditions.

14. The method according to claim 1, wherein the taggant sample is applied to the item in a manner that allows removal of a sample for identification.

15. The method according to claim 14, wherein the taggant sample is ink.

16. The method according to claim 15, wherein the taggant sample is printed onto the item or a package containing the item.

17. The method according to claim 1, wherein the item to which the taggant sample is applied is selected from the group consisting of fabric, paper, cardboard, wood, plastic, nylon, nitrocellulose, rubber, resin, gel, liquid, and adhesive.

18. The method according to claim 17, wherein the item is fabric.

19. The method according to claim 18, wherein the item is an article of clothing.

20. The method according to claim 17, wherein the item is paper or plastic.

21. The method according to claim 19, wherein the paper or plastic is a label.

22. The method according to claim 21, wherein the label is a bar-code label.

23. The method according to claim 21, wherein the label is a tamper-proof label.

24. The method according to claim 17, wherein the item is cardboard.

25. The method according to claim 24, wherein the cardboard is a product's packaging.

26. The method according to claim 25, wherein the taggant is applied to the exterior surface of the packaging.

27. The method according to claim 24, wherein the taggant is incorporated into the product's packaging.

28. The method according to claim 1, wherein the item the taggant is applied to a medicament.

29. The method according to claim 28, wherein the medicament is selected from the group consisting of a capsule, a pill, a tablet, a lozenge, and an ointment.

30. The method according to claim 1, wherein the device has a plurality of pairs of separated electrical conductors, each pair having attached capture probes that are complementary to a different target nucleic acid.

31. A nucleic acid taggant identification kit comprising:
a nucleic acid-containing taggant comprising one or more target nucleic acids and
a detection cartridge comprising:
one or more sets of electrically separated electrical conductor pairs, each conductor having an attached capture probe such that a gap exists between the capture probes of a pair of electrically separated conductors, wherein the capture probes for each pair of separated electrical conductors are complementary to one of the target nucleic acids.

32. The kit according to claim 31, wherein the target nucleic acids are selected from the group consisting of DNA, RNA, peptide nucleic acids, and locked nucleic acids.

33. The kit according to claim 31, wherein the taggant sample further comprises random nucleic acid molecules mixed with the target nucleic acids.

34. The kit according to claim 31, wherein the target nucleic acids comprise 10 to 30 nucleotides.

35. The kit according to claim 31, wherein the taggant mixture further comprises a matrix.

36. The kit according to claim 35, wherein the matrix is selected from the group consisting of polyvinylalcohol, polyethyleneglycol, polyethyleneimine, polyvinylpyridine, hydroxyethylcellulose, polyvinylbutyral, polyvinylpyrrolidone, polyvinylimidazole, and a combination thereof.

37. The kit according to claim 31 further comprising:
a material capable of producing a conductive coating.
